(19) United States
(12) Patent Application Publication
(10) Pub. No.: US 2008/0015260 A1
(43) Pub. Date: Jan. 17, 2008

(54) Pharmaceutical compositions having improved stability and methods for preparation and use

(75) Inventors: Jeffrey S. Kiel, Gainesville, GA (US); H. Gregory Thomas, Carrolton, GA (US); Richard Andrew Todebush, Norcross, GA (US); Amy O. Barish, Cumming, GA (US)

Correspondence Address: Marla J. Church, Esq.;Kiel Laboratories, Inc., 2225 Centennial Drive, Gainesville, GA, 30504, US

(73) Assignee: Kiel Laboratories, Inc.

(21) Appl. No.: 11/827,363

(22) Filed: Jul. 11, 2007

Related U.S. Application Data
(60) Provisional application No. 60/830,501, filed on Jul. 13, 2006.

(51) Int. Cl. A61K 31/133

(52) U.S. Cl. 514/653

(57) ABSTRACT

The present invention relates to novel compositions having improved stability during storage over extended periods of time. The compositions of the present invention comprise an active pharmaceutical ingredient combined with a synthetic form of magnesium aluminum silicate. The present invention also relates to novel compositions that comprise active pharmaceutical ingredients as tannate complexes combined with a synthetic form of magnesium aluminum silicate wherein the compositions have an increased weight percent of insoluble active pharmaceutical ingredient. The present invention also relates to a method for preparing the novel compositions.

This application claims the benefit of U.S. Provisional Patent Application Ser. No. 60/830,501 filed on Jul. 13, 2006.

### FIELD OF THE INVENTION

The present invention relates to novel compositions of active pharmaceutical ingredients having improved stability during storage over extended periods of time in liquid or tablet form, which retain significant pharmaceutical activity during such storage. The present invention also relates to novel pharmaceutical suspensions and tablets having insoluble tannate complexes with improved sustained release characteristics. The present invention is further directed to methods for preparing such pharmaceutical compositions and methods of use.

### BACKGROUND OF THE INVENTION

There are many prescription and non-prescription, or “over the counter”, medications or pharmaceutical compositions available for use. Compositions for oral use include accepted formulations such as liquids or suspensions, tablets, and capsules. These compositions generally include many ingredients, one or more of which are typically active ingredients. An active pharmaceutical ingredient (API) is a substance having a specific physiological action with minimal or no side effects. While pharmaceutical compositions may have a single API, it is often desirable to combine multiple API's in a single pharmaceutically acceptable composition. For example, a single medication including multiple active ingredients may be administered for treating allergy symptoms, such as a runny nose, a fever, a cough, and inflammation wherein each active ingredient is directed towards a specific allergy symptom. Whenever a composition has a combination of pharmaceutical ingredients, whether active or non-active, the stability of the composition may be compromised either because the single API is inherently unstable or the inclusion of a combination of two or more active ingredients and/or the presence of non-active ingredients, i.e., excipients, may reduce stability.

Stability issues may adversely affect the cost of commercializing the composition. Before a formulated composition or medication may be made commercially available for public use in the United States, it must pass strict stability standards set forth by the United States Food and Drug Administration (FDA) and the United States Pharmacopeia (USP). Particularly, the FDA requires that a medication has a certain level of stability over a period of time. More specifically, the ingredients in a composition collectively need to remain stable thereby maintaining the overall activity, function and therefore, safety of the composition for that specified period of time. Improved stability is important to extend the shelf life of any particular pharmaceutical composition while complying with both USP and FDA requirements. It is therefore desirable to improve the stability of pharmaceutical compositions to comply with government stability requirements.

Pharmaceutical compositions must undergo extensive testing in order to ensure safety and efficacy. A portion of the testing is performed in order to establish an expiration date. Products must be labeled with an expiration date that identifies the time past which the safety and efficacy cannot be guaranteed due to degradation of the API. As API's degrade, the effective amount of the API diminishes and by-products of the degradation build up. The resulting reduction in the amount of API renders the product less effective, and resulting degradation by-products can contribute to adverse reactions in the patient taking the product.

Many factors can contribute to the degradation of the pharmaceutical product including increased storage temperature, exposure to light and adverse interactions between the active ingredient and excipients. Thus, there is a need to improve the stability of pharmaceutical compositions. There is also a need to do so in a manner that is mild and non-destabilizing for the API. Further, there is a need to improve the overall stability and shelf life of the composition sufficient to meet or exceed government standards. Still further there is a need to provide this composition having improved stability in a convenient and cost effective manner.

It is known that tannate salts of API's provide therapeutic activity over longer periods of time. By providing an API in a tannate salt form one extends the release profile of the API. Consequently, the API in a tannate salt form does not need to be dosed as frequently as conventional dosage forms. There is a need to provide an API/tannate dosage form having improved conversion to the API/tannate complex for selected API's that have exhibited a less than desired percent conversion of active pharmaceutical ingredient to API/tannate complex.

### SUMMARY OF THE INVENTION

The present invention provides compositions with improved stability, methods of preparing the compositions and methods of using the improved stable compositions. One factor that can contribute to the degradation of the pharmaceutical product is adverse interactions between the excipients and the active ingredient. Magnesium Aluminum Silicate (MAS) is commonly used as a pharmaceutical excipient, often as a dispersing agent. MAS also possesses binder properties. MAS is typically mined and processed from natural sources which, even after extensive processing, contains many impurities. Of particular interest are heavy metal ions which may promote chemical reactions leading to the degradation of the API. Recently, synthetic forms of MAS have become available. One commercially available form of synthetic MAS is manufactured and sold by Fugi Chemical of Japan under the trade name Neusilin®. The specific grade used in the examples is UFL2. The synthetic forms of MAS contain fewer heavy metal ion impurities. In accordance with the present invention, it has been discovered that the use of synthetic MAS as an excipient in a pharmaceutical composition dramatically increases the stability of certain API(s) resulting in a product with a longer shelf life and also with fewer impurities.

In accordance with the present invention it has been discovered that the use of synthetic MAS as an excipient in an API/tannic acid reaction mixture significantly improves the conversion rate to an API/tannate complex, as measured by the wt % of insoluble API.

### BRIEF DESCRIPTION OF THE DRAWINGS

FIG. 1 shows the conversion of phenylephrine to the tannate complex in the presence of Neusilin® at various concentrations compared to the presence of MAS.

FIG. 2 shows the conversion of carbetapentane to the tannate complex in the presence of Neusilin® at various concentrations compared to the presence of MAS.

FIG. 3 shows the conversion of chlorpheniramine to the tannate complex in the presence of Neusilin® at various concentrations compared to the presence of MAS.

### DETAILED DESCRIPTION OF THE INVENTION

The present invention is based upon the identification and preparation of novel compositions utilizing Neusilin® which are stable when stored as liquids or tablets for extended periods of time, which do not lose significant pharmaceutical activity during such storage and/or which are pharmaceutically acceptable in that they are well tolerated when administered. More specifically, the present invention is directed to improving the stability of API's in tannate compositions. The present invention is also based upon the improved conversion rate to an API/tannate complex as measured by the wt % of insoluble API. The present invention is also directed to methods for preparing such compositions, methods for enhancing the stability of the active ingredient in such compositions, methods of improving tannate complex conversion rate for selected active agents and methods of using such compositions for administering various therapeutic categories of active pharmaceutical ingredients such as antihistamines, antiemetics, antinausea agents, antitussives, anticholinergics and decongestants to a patient in need of such treatment.

The novel compositions of the present inventions comprise, in part, the presence of Neusilin®. The amount of Neusilin® recommended in the formulation ranges from approximately 0.1-10.0% for suspensions and 0.1-50.0% for solid dosage forms. The amount of Neusilin® required in the formulation to improve rate of conversion to an API/tannate complex varies from approximately 0.25%-8% (as set forth in the Examples).

The novel compositions of the present invention further comprise, in part, a therapeutically effective amount of API. “Therapeutically effective amount” means that the composition contains a concentration of API which when administered to an individual in need of such treatment, results in a desired pharmacological therapeutic effect.

The present invention may be illustrated by example. The following Examples illustrate specific embodiments of the present invention. These Examples are only intended to illustrate the present invention and are not intended to limit the scope thereof. Many variations in composition may be made without departing from the spirit and scope of the present invention.

### EXAMPLE 1

A comparative study was carried out to evaluate MAS versus Neusilin® with regard to the stability of phenylephrine HCl and chlorpheniramine maleate in a suspension formulation containing each active ingredient as a tannate salt complex prepared according to the method disclosed in U.S. Pat. No. 6,869,618 (the '618 patent) which is incorporated herein by reference. The MAS used in all examples was VEEGUM® which is a registered trademark owned by R. T. Vanderbilt Company, Inc. Two compositions were prepared and analyzed separately. Composition A contained MAS and Composition B contained Neusilin® (NEU). Each composition contained 2.5 mg/5 mL phenylephrine HCl and 2.25 mg/5 mL of chlorpheniramine maleate in the finished tannate suspension product. These compositions are defined as follows for a batch size of 5000 mL:

Composition A: MAS

| | % in total |
|---|---|
| Raw Material | (w/v) |
| Phenylephrine HCl | 0.04 |
| Chlorpheniramine Maleate | 0.056 |
| Tannic Acid, USP | 0.12 |
| Magnesium Aluminum Silicate, NF | 0.80 |
| Citric Acid | 0.44 |
| Sodium Saccharin | 0.30 |
| Sucrose, NF | 10.00 |
| Glycerin, USP | 7.50 |
| Sodium Citrate Dihydrate, USP | 1.00 |
| Methylparaben | 0.20 |
| Sodium Benzoate, NF | 0.10 |
| Xanthan Gum | 0.52 |
| Grape Flavor | 0.20 |
| FD&C Blue #1 | 0.002 |
| FD&C Red #40 | 0.005 |
| Purified Water, USP | N/A |

Composition B: NEUSILIN®

| Raw Material | % (w/v) |
|---|---|
| Phenylephrine HCl | 0.04 |
| Chlorpheniramine Maleate | 0.056 |
| Tannic Acid, USP | 0.12 |
| Neusilin ®, NF | 0.80 |
| Citric Acid | 0.44 |
| Sodium Saccharin | 0.30 |
| Sucrose, NF | 10.00 |
| Glycerin, USP | 7.50 |
| Sodium Citrate Dihydrate, USP | 1.00 |
| Methylparaben | 0.20 |
| Sodium Benzoate, NF | 0.10 |
| Xanthan Gum | 0.52 |
| Grape Flavor | 0.20 |
| FD&C Blue #1 | 0.002 |
| FD&C Red #40 | 0.005 |
| Purified Water, USP | N/A |

The assay results for chlorpheniramine maleate and phenylephrine HCl in samples stored at 25° C. and 60% Relative Humidity (RH) are shown in Table 1. After 12 months, Composition A with MAS showed a loss of phenylephrine at 11.9% while Composition B with Neusilin® showed no decrease (NC) in phenylephrine. With regard to chlorpheniramine, Composition A with MAS showed an increase of chlorpheniramine at 4.2% while Composition B with Neusilin® showed no increase or decrease in chlorpheniramine. The increase in chlorpheniramine remains within the acceptable range of 90.0-110.0% Label Claim (LC).

TABLE 1

| | Chlorpheniramine | Phenylephrine |
|---|---|---|
| Composition A: MAS | +4.2% | −11.9% |
| Composition B: NEU | NC | NC |

### EXAMPLE 2

A comparative study was carried out to evaluate MAS versus Neusilin® with regard to the stability of diphenhydramine HCl and phenylephrine HCl in a chewable tablet formulation containing each active ingredient as a tannate salt complex prepared according to U.S. 2003/0077321 (the '321 publication) which is incorporated herein by reference. Two compositions were prepared and analyzed separately. Composition C contained MAS and Composition D contained Neusilin® (NEU). The compositions each contained 12.5 mg/500 mg diphenhydramine HCl and 5.0 mg/500 mg phenylephrine HCl in the finished tannate tablet product. These compositions are defined as follows for a 5000.00 gm batch size:

Composition C: MAS

| | % in total |
|---|---|
| Raw Material | (w/w) |
| Diphenhydramine HCl | 2.50 |
| Phenylephrine HCl | 1.00 |
| Tannic Acid, USP | 3.50 |
| Magnesium Aluminum Silicate, NF | 1.35 |
| Mannitol | 29.56 |
| Sodium Saccharin | 0.25 |
| Magnasweet ® ¹ | 1.50 |
| Corn Starch | 1.00 |
| Methocel ® E-10M ² | 1.35 |
| Di-Pac ® ³ | 50.00 |
| Calcium Phosphate Dibasic | 2.70 |
| Citric Acid | 2.00 |
| Strawberry Flavor | 1.20 |
| FD&C Blue #1 | 0.09 |
| Talc, USP | 1.00 |
| Magnesium Stearate, NF | 1.00 |
| Purified Water, USP | N/A |

¹ Magnasweet ® is a registered trademark of Mafco Worldwide Corporation.
² Methocel ® is a registered trademark of Dow Chemical Company.
³ Di-Pac ® is a registered trademark of American Sugar Refining, Inc.

Composition D: NEUSILIN®

| | % in total |
|---|---|
| Raw Material | (w/w) |
| Diphenhydramine HCl | 2.50 |
| Phenylephrine HCl | 1.00 |
| Tannic Acid, USP | 3.50 |
| Neusilin ®, NF | 1.35 |
| Mannitol | 29.56 |
| Sodium Saccharin | 0.25 |
| Magnasweet ® | 1.50 |
| Corn Starch | 1.00 |
| Methocel ® E-10M | 1.35 |
| Di-Pac ® | 50.00 |
| Calcium Phosphate Dibasic | 2.70 |
| Citric Acid | 2.00 |
| Strawberry Flavor | 1.20 |
| FD&C Blue #1 | 0.09 |
| Talc, USP | 1.00 |
| Magnesium Stearate, NF | 1.00 |
| Purified Water, USP | N/A |

The assay results for diphenhydramine HCl and phenylephrine HCl in samples stored at 60° C. ambient RH are shown in Table 2. The RH is not measured for samples stored at 60° C. . After 4 weeks, Composition C with MAS showed a loss of phenylephrine HCl at 21.1% while Composition D with Neusilin® showed a decrease of 12.1%. With regard to diphenhydramine HCl, Composition C with MAS showed a decrease of 10.4% while Composition D with Neusilin® showed a decrease of 4.1%.

TABLE 2

| | Diphenhydramine | Phenylephrine |
|---|---|---|
| Composition C: MAS | −10.4% | −21.1% |
| Composition D: NEU | −4.1% | −12.1% |

### EXAMPLE 3

A comparative study was carried out to evaluate MAS versus Neusilin® with regard to the stability of brompheniramine maleate and phenylephrine HCl in a tannate salt complex suspension. Two compositions were prepared according to the '618 patent and analyzed separately. Composition E contained MAS and Composition F contained Neusilin® (NEU). The compositions each contained 6 mg/5 mL brompheniramine maleate and 10 mg/5 mL phenylephrine HCl in the finished suspension product. These compositions are defined as follows for a batch size of 5000 mL:

Composition E: MAS

| | % in total |
|---|---|
| Raw Material | (w/v) |
| Brompheniramine Maleate, USP | 0.12 |
| Phenylephrine HCl, USP | 0.20 |
| Tannic Acid, USP | 0.32 |
| Magnesium Aluminum Silicate, NF | 0.80 |
| Citric Acid, USP | 0.50 |
| Sucrose, NF | 25.00 |
| Sodium Saccharin, USP | 0.10 |
| Glycerin, USP | 7.50 |
| Sodium Citrate Dihydrate, USP | 1.00 |
| Methylparaben, NF | 0.20 |
| Sodium Benzoate, NF | 0.10 |
| Xanthan Gum | 0.42 |
| Artificial Bubble Gum Flavor | 1.000 |
| FD&C Red #40 | 0.008 |
| Purified Water, USP | N/A |

Composition F: NEUSILIN®

| | % in total |
|---|---|
| Raw Material | (w/v) |
| Brompheniramine Maleate, USP | 0.12 |
| Phenylephrine HCl, USP | 0.20 |
| Tannic Acid, USP | 0.32 |
| Neusilin ®, NF | 0.80 |
| Citric Acid, USP | 0.50 |
| Sucrose, NF | 25.00 |
| Sodium Saccharin, USP | 0.10 |
| Glycerin, USP | 7.50 |
| Sodium Citrate Dihydrate, USP | 1.00 |
| Methylparaben, NF | 0.20 |
| Sodium Benzoate, NF | 0.10 |
| Xanthan Gum | 0.42 |
| Artificial Bubble Gum Flavor | 1.000 |
| FD&C Red #40 | 0.008 |
| Purified Water, USP | N/A |

The assay results for brompheniramine maleate and phenylephrine HCl in samples stored at 25° C. and 60% RH are shown in Table 3. After 3 months, Composition E with MAS showed a loss of brompheniramine maleate at 2.5% while Composition F with Neusilin® showed no decrease (NC). With regard to phenylephrine HCl, Composition E with MAS showed a decrease of 6.5% while Composition F with Neusilin® showed no decrease (NC).

TABLE 3

| | Brompheniramine | Phenylephrine |
|---|---|---|
| Composition E: MAS | −2.5% | −6.5% |
| Composition F: NEU | NC | NC |

### EXAMPLE 4

The following example is a tannate tablet formulation containing 15 mg carbetapentane citrate, 12.5 mg of diphenhydramine HCl and 5 mg phenylephrine HCl per each tablet.

The tannate salt complex was prepared using the method disclosed in the '321 publication. The tablets were prepared using techniques known in the art. Tablets prepared in this way exhibit a better stability profile than those prepared using MAS derived from a natural source in the place of Neusilin®. The effect is particularly pronounced in relation to phenylephrine. The composition is defined as follows:

| | % W/W in |
|---|---|
| Raw Material | Formulation |
| Diphenhydramine HCl | 2.50 |
| Phenylephrine HCl | 1.00 |
| Carbetapentane Citrate | 3.00 |
| Tannic Acid, USP | 6.50 |
| Neusilin ®, NF | 1.35 |
| Corn Starch | 1.00 |
| Methocel ® E-10M | 1.35 |
| Avicel ® PH 102¹ | 77.78 |
| Calcium Phosphate Dibasic | 2.70 |
| Xanthan Gum | 1.58 |
| Talc, USP | 0.25 |
| Magnesium Stearate, NF | 1.00 |
| Purified Water, USP | N/A |

¹Avicel ® is a registered trademark of FMC Corporation.

### EXAMPLE 5

The following example is a tablet formulation containing 5 mg phenylephrine HCl per each tablet.

The tablets can be prepared using techniques known in the art. Tablets prepared in this way exhibit a better stability profile for phenylephrine than those prepared using MAS derived from a natural source. The composition is defined as follows:

| | % W/W in |
|---|---|
| Raw Material | Formulation |
| Phenylephrine HCl | 1.00 |
| Neusilin ®, NF | 1.35 |
| Corn Starch | 1.00 |
| Methocel ® E-10M | 1.35 |
| Avicel ® PH 102 | 89.78 |
| Calcium Phosphate Dibasic | 2.70 |
| Xanthan Gum | 1.58 |
| Talc, USP | 0.25 |
| Magnesium Stearate, NF | 1.00 |
| Purified Water, USP | N/A |

### EXAMPLE 6

The following example is a suspension formulation containing phenylephrine HCl/tannate salt complex. The tannate salt complex was prepared according to the '618 patent. The final suspension was prepared using techniques known in the art. Suspensions prepared in this way exhibit a better stability profile than those prepared using MAS derived from a natural source. The composition is defined as follows:

| | % W/V in |
|---|---|
| Raw Material | Formulation |
| Phenylephrine HCl | 0.08 |
| Tannic Acid, USP | 0.08 |
| Neusilin ®, NF | 0.80 |
| Sodium Phosphate Dibasic | 0.65 |
| Sucrose | 10.00 |
| Sodium Saccharin | 0.30 |
| Glycerin USP | 20.00 |
| Sodium Phosphate Monobasic | 0.40 |
| Methylparben, NF | 0.20 |
| Sodium Benzoate, NF | 0.10 |
| Xanthan Gum | 0.62 |
| Sweet Tangerine Flavor | 0.70 |
| FD&C Yellow #6 | 0.015 |
| Purified Water | N/A |

### EXAMPLE 7

The following example is a series of reaction mixtures for suspension formulations containing phenylephrine HCl, tannic acid and MAS or various concentrations of Neusilin®:

| | Wt % of Total |
|---|---|
| Ingredient | Suspension |
| Phenylephrine HCl | 3.5% |
| Tannic Acid | 8.7% |
| MAS | 8.7% |
| Neusilin ® | 8.7% |
| ½ Neusilin ® | 4.4% |
| ¼ Neusilin ® | 2.2% |

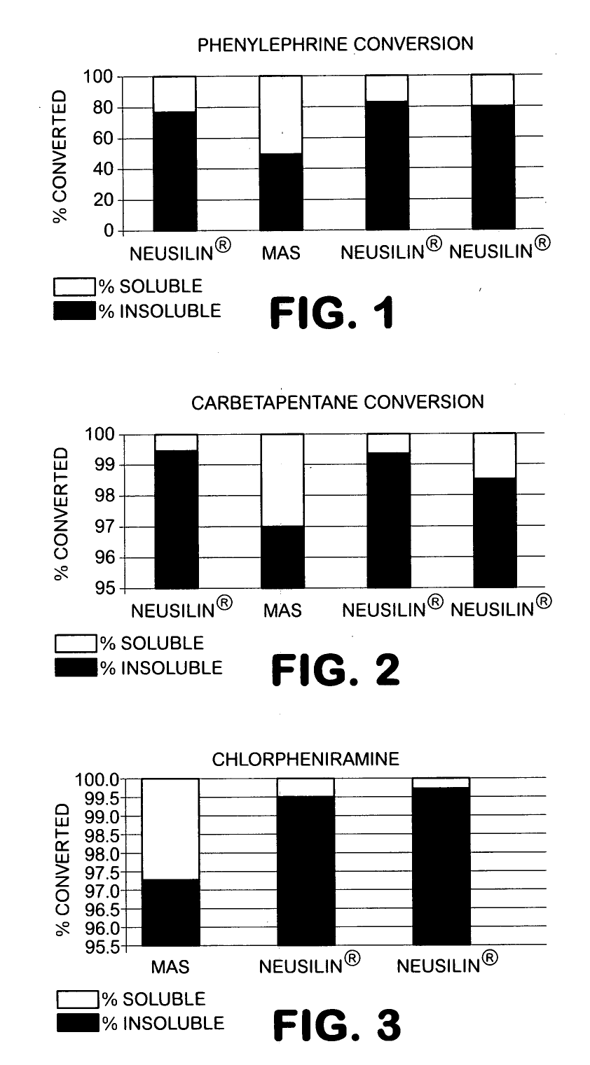

Analysis of the above suspensions was carried out by removing 10 gms of product and centrifuging. The amount of API found in the insoluble portion of the suspension was reported as the % converted insoluble and the liquid portion as the % converted soluble. The results for phenylephrine HCl are shown in FIG. 1.

The data demonstrate that the conversion of phenylephrine to the tannate complex was greatly increased, >30%, with the presence of Neusilin® in the suspension. The data in FIG. 1 also show that the use of Neusilin® at levels ½ to ¼ showed the same increase in conversion of phenylephrine to the tannate complex.

### EXAMPLE 8

The following example is a series of reaction mixtures for suspension formulations containing carbetapentane citrate, tannic acid and MAS or various concentrations of Neusilin®:

| | Wt % of Total |
|---|---|
| Ingredient | Suspension |
| Carbetapentane Citrate | 3.5% |
| Tannic Acid | 8.7% |
| MAS | 8.7% |
| Neusilin ® | 8.7% |
| ½ Neusilin ® | 4.4% |
| ¼ Neusilin ® | 2.2% |

Analysis of the above suspensions was carried out by removing 10 gms of product and centrifuging. The amount of API found in the insoluble portion of the suspension was reported as the % converted insoluble and the liquid portion as the % converted soluble. The results for carbetapentane citrate are shown in FIG. 2.

The conversion data for carbetapentane, as demonstrated in FIG. 2, show that the use of Neusilin® does not significantly affect the level of conversion when compared to MAS. It is interesting that the use of Neusilin® at reduced concentrations of ½ or ¼ showed the same level of conversion.

### EXAMPLE 9

The following example is a series of reaction mixtures for suspension formulations containing chlorpheniramine maleate, tannic acid and MAS or various concentrations of Neusilin®:

| | Wt % of Total |
|---|---|
| Ingredient | Suspension |
| Chlorpheniramine Maleate | 3.5% |
| Tannic Acid | 8.7% |
| MAS | 8.7% |
| Neusilin ® | 8.7% |
| ½ Neusilin ® | 4.4% |
| ¼ Neusilin ® | 2.2% |

Analysis of the above suspension was carried out by removing 10 gms of product and centrifuging. The amount of API found in the insoluble portion of the suspension was reported as the % converted insoluble and the liquid portion as the % converted soluble. The results for chlorpheniramine maleate are shown in FIG. 3.

As seen with carbetapentane in Example 8, the conversion data for chlorpheniramine show that the use of Neusilin® does not significantly affect the level of conversion when compared to MAS. It is also interesting, as observed with carbetapentane, that the use of Neusilin® at a reduced concentration of ¼ showed the same level of conversion as the ½ reduction.

The assays for each active ingredient in the above examples were performed according to standard protocols known in the art.

The foregoing description of the preferred embodiments of the present invention has been presented for purposes of illustration and description. They are not intended to be exhaustive or to limit the invention to the precise form disclosed, and many modifications and variations are possible in light of the above teaching. Such modifications and variations which may be apparent to a person skilled in the art are intended to be within the scope of the invention.

What is claimed:

1. A pharmaceutical composition having improved stability comprising at least one active pharmaceutical ingredient and a synthetic form of magnesium aluminum silicate.

2. The composition of claim 1 wherein the at least one active pharmaceutical ingredient is phenylephrine and/or its salts.

3. The composition of claim 2 wherein the phenylephrine is phenylephrine hydrochloride.

4. The composition of claim 2 wherein the phenylephrine is phenylephrine tannate.

5. The composition of claim 4 wherein the composition is in a solid dosage form.

6. The composition of claim 4 wherein the composition is in a liquid dosage form.

7. The composition of claim 5 wherein the synthetic magnesium aluminum silicate is present in a range of 0.1-10% weight/weight.

8. The composition of claim 4 wherein the synthetic magnesium aluminum silicate is present in a range of 0.1-10% weight/volume.

9. A method for improving the stability of a pharmaceutical composition comprising the steps of:
providing at least one active pharmaceutical ingredient; and
mixing a salt or free base of the at least one active pharmaceutical ingredient with a synthetic magnesium aluminum silicate in a pharmaceutically acceptable medium.

10. The method of claim 9 wherein the at least one active pharmaceutical ingredient is phenylephrine and/or its salts.

11. The method of claim 10 wherein the phenylephrine is phenylephrine hydrochloride.

12. The method of claim 11 further comprising adding tannic acid to the mixing step.

13. A pharmaceutical tannate composition comprising an active pharmaceutical agent, tannic acid and a synthetic form of magnesium aluminum silicate, said composition having an increased weight percent of insoluble active pharmaceutical agent.

14. The tannate composition of claim 13 wherein the active pharmaceutical ingredient is phenylephrine.
